(19) United States
(12) Patent Application Publication
(10) Pub. No.: US 2008/0312129 A1
(43) Pub. Date: Dec. 18, 2008

(54) Use of Mas G-Protein-Coupled Receptor Agonists and Antagonists, as Apoptotic-Activity Modulators for Study, Prevention and Treatment of Diseases

(76) Inventors: Robson Augusto Souza Dos Santos, Belo Horizonte-MG (BR); Sergio Veloso Brant Pinheiro, Nova Lima-MG (BR); Raphael De Faria E Silva, Belo Horizonte-MG (BR); Ivana Silva Lula, Belo Horizonte-MG (BR); Frederico Barros De Sousa, Belo Horizonte-MG (BR); Frederic Jean Georges Frezard, Belo Horizonte-MG (BR); Adelina Martha Dos Reis, Belo Horizonte-MG (BR); Luiz Renato De Franca, Belo Horizonte-MG (BR); Anderson Jose Ferreira, Belo Horizonte-MG (BR); Rubert Dario Sinisterra, Belo Horizonte-MG (BR); Maria Jose Campagnole Santos, Belo Horizonte-MG (BR); Walkyrie Neyde De Oliveira Sampaio, Belo Horizonte-MG (BR); Marcelo De Castro Leal, Belo Horizonte-MG (BR)

Correspondence Address: NIXON & VANDERHYE, PC, 901 NORTH GLEBE ROAD, 11TH FLOOR, ARLINGTON, VA, 22203, US

(21) Appl. No.: 11/922,949

(22) PCT Filed: Jun. 28, 2006
(86) PCT No.: PCT/BR2006/000125

§ 371 Date: Aug. 13, 2008

(30) Foreign Application Priority Data
Jun. 28, 2005 (BR) PI0502497-8

(51) Int. Cl. A61K 38/08; A61P 21/00; A61K 48/00; A61P 9/00

(52) U.S. Cl. 514/2; 514/44

(57) ABSTRACT

The present invention is characterized by the use of Mas G-protein-coupled receptor agonists and antagonists as apoptotic activity modulators for study, prevention and treatment of diseases. It is further characterized by the use of Mas G-protein-coupled receptor agonist and antagonists for modulation of the apoptotic activity involving alterations in the activity of the protein kinase B/Akt. Another characteristic of the invention is the use of Mas G-protein-coupled receptor agonists and antagonists, including the Ang-(1-7) peptide and its analogues, agonists and antagonists, either peptidic or non-peptidic, as modulators of apoptotic activity for study, prevention and treatment of degenerative diseases of organs and systems, as an auxiliary measure for organs transplantation, treatment with embryonic, non-embryonic stem cells, re-implantation of organs and tissues and other treatments that need temporary or chronic reduction of the apoptotic activity, not limitative. The invention further claims the use of Mas G-protein-coupled receptor agonists and antagonists, including the Angiotensin-(1-7) peptide and its analogues, agonists and antagonists, either peptidic or non-peptidic, formulated with pharmaceutically and pharmacologically acceptable excipients or carriers, as modulators of the apoptotic activity.

The present invention is characterized by the use of Mas, G-protein-coupled receptor agonists and antagonists, as apoptotic-activity modulators for study, prevention and treatment of diseases.

The invention is further characterized by the use of Mas, G-protein-coupled receptor agonists and antagonists, for modulation of apoptotic activity involving alterations of the activity of the B/Akt kinase protein.

Another characteristic of the invention is the use of Mas, G-protein-coupled receptor agonists and antagonists, including the angiotensin-(1-7) peptide and analogs, agonists and antagonists thereof, either peptidic or non-peptidic, as apoptotic-activity modulators for use in the study, prevention and treatment of diseases.

The invention further claims the use of Mas, G-protein-coupled receptor agonists and antagonists, formulated with pharmaceutically or pharmacologically acceptable carriers, and Mas, G-protein-coupled receptor agonists and antagonists, including the angiotensin-(1-7) peptide and analogs, agonists and antagonists thereof, either peptidic or non-peptidic, as apoptotic-activity modulators.

Another claimed feature is the use of micro- and nanoparticulate, implantable or injectable devices of formulations of the Mas, G-protein-coupled receptor agonists and antagonists, including the antiotensin-(1-7) peptide and analogs, agonists and antagonists thereof, either peptidic or non-peptidic, as apoptotic-activity modulators.

The presently described administration forms contain but are not limited to the use of Mas, G-protein-coupled receptor agonists and antagonists, including the angiotensin-(1-7) peptide and analogs, agonists and antagonists thereof, either peptidic or non-peptidic, and formulations thereof for use through the oral, intramuscular, endovenous, subcutaneous, topical, transdermic, anal, inhalation (pulmonary, intranasal, intrabuccal) administration routes or as devices that could be implanted or injected for the study, prevention and treatment of diseases.

The role of the Renin-Angiotensin-System (RAS) as a regulator of homeostasis of body liquids and of blood pressure is quite known. The RAS is responsible for the regulation of blood pressure, cardiovascular homeostasis and of the hydroelectrolytic balance, in both physiological and pathological conditions (Santos, R. A. S.; Campagnole-Santos, M. J.; Andrade, S. P. Angiotensin-(1-7): an update. Regul Pept. 91:45-62, 2000). Recently, it was found that, besides the system that generates Ang II in the blood circulation, different tissues can generate various biologically active peptides of this system locally (tissular RAS). The components of the tissular RAS are found in various organs and tissues, including the heart, vessels, kidney, the male and female reproductive system, endocrinal glands, bone cord and brain. The functions of these RAS's in different tissues still are not completely clarified (Santos, R A S, Campagnole-Santos, M J, Andrade, S P, Angiotensin-(1-7): an update. Regul Pept. 91:45-62, 2000; Yoshimura, Y. The ovarian rennin-angiotensin system in reproductive physiology. Front Neuroendocrinol.; 18: 247-291, 1997).

The primary components of the classic RAS are: Renin, the enzyme that catalyzes the proteolytic conversion of angiotensinogen into Angiotensin I (Ang I); angiotensinogen, the main substrate of rennin and precursor of Angiotensin II (AngII); the Angiotensin converting enzyme (ACE), which converts Ang I into Ang II by hydrolysis of the two carboxyterminal amino acids; Angiotensin II, the main biologically active peptide of the system; and the AT₁ and AT₂ receptors, responsible for the initiation of the cellular effects of Ang II. To this well-known system, other peptides have been added, such as Angiotensin III (Ang III), Angiotensin IV (Ang IV) and Angiotensin-(1-7) [(Ang-(1-7))]. Ang-(1-7) can be generated from Ang I or Ang II by tissular endopeptidases (Santos, R. A., Brosnihan, K. B., Cappell, M. C., Pesquero, J., Chernicky, C. L., Greene, L. J.; Ferrario, C. M. Converting enzyme activity and angiotensin metabolism in the dog brainstem. Hypertension (11(2-Pt 2): I153-I1537, 1988).

Ang-(1-7) has been identified in the plasma and in various human and animal organs and tissues (Santos, R. A. S.; Campagnole-Santos, M. J.; Andrade, S. P. Angiotensin-(1-7): an update. Regul Pept 91:45-62, 2000), including female rat ovaries (Costa A P R, Fagundes-Moura C R, Pereira V M, Silva L F, Vieira M A, Santos R A, Reis A M). Angiotensin-(1-7): a novel peptide in the ovary. Endocrinology. 144:1942-1948, 2003). Its independent production of ACE was demonstrated by Santos et al., 1988 (Santos, R. A., Brosnihan, K. B., Cappell, M. C., Pesquero, J., Chernicky, C. L., Greene, L. J.; Ferrario, C. M. Converting enzyme activity and angiotensin metabolism in the dog brainstem. Hypertension. 11 (2-Pt 2): I153-I153-I1537, 1988) and its attachment to a specific receptor suggested by Santos et al., 1994 (Santos, R A S, Campagnole-Santos, M. J., Baracho, N. C., Fontes, M. A., Silva, L. C., Neves, L. A. , Oliveira, D. R., Caligiorne, S. M., Rodrigues, A . R., Gropen Junior, C. Characterization of a new angiotensin antagonist selective for angiotensin-(1-7): evidence that the actions of angiotensin-(1-7) are mediated by specific angiotensin receptors. Brain Res Bull. 35(4):293-298, 1994) and Tallant et al., 1997, (Tallant, E. A, Lu, X., Weiss, R. B., Cappell, M. C.; Ferrario, C. M. Bovine aortic endothelial cells contain an angiotensin-(1-7) receptor. Hypertension 29:388-393, 1997). These findings have been obtained especially as a result of the availability of a selective antagonist for Ang-(1-7), the A-779 (Asp¹-Arg²-Val³-Tir⁴-Lle⁵-His⁶-D-Ala⁷; Santos, R. A ., Campagnole-Santos, M. J. Barracho, N. C., Fontes, M. A, Silva, L. C., Neves, L. A, Oliveira, D. R., Caligione, S. M., Rodrigues, A. R., Groppen Junior, C. Characterization of a new angiotensin antagonist selective for angiotensin-(1-7): evidence that the actions of angiotensin-(1-7) are mediated by specific angiotensin receptors. Brain Res Bull. 35(4):293-298, 1994; Ambuhl, P, Felix, D. Khosla, M. C. [7-D-ALA]-Angiotensin-(1-7): selective antagonism of angiotensin-(1-7) in the rat paraventricular nucleus. Brain Res Bull. 1994; 35(4):289-91).

Various enzymes are involved in several steps of the RAS cascade, where they are responsible for the degradation of some peptidic fragments, as well as for the generation of others. The ACE is responsible for converting Ang I into Ang II; the PEP (Prolyl-endopeptidase) generates Ang-(1-7) from Ang I and Ang II, and NEP (Neutral Endopeptidase) catalyzes the conversion of Ang I into An-(1-7). The ACE also hydrolyzes Ang-(1-7), generating Ang-(1-5) (Chappell M C, Pirro N T, Sykes, A, Ferrario C M. Metabolism of angiotensin-(1-7) by angiotensin-converting enzyme. Hypertension. 31:362-367, 1998). Thus, the ACE pathway is important for both generation and degradation of circulating and tissular biologically active peptides of the RAS. More recently, another enzyme was described, which is important for the formation of Ang-(1-7), the ACE2 (S R Tipnis, N M Hooper, R Hyde, E Karran and G Christie. The human homolog of angiotensin-converting enzyme. Cloning and functional expression as a captopril-insensitive carboxypeptidase. J. Biol Chem. 275(43):33238-33243, 200). This enzyme forms Ang-(1-7), especially from Ang II.

Angiotensin-(1-7) and angiotensin II are the main RAS effectors. Two important characteristics differentiate Ang-(1-7) from Ang II: first Ang-(1-7) has highly specific biological actions and according to the pathway of formation of Ang-(1-7) can be completely independent of ACE (Santos, R. A. S.; Campagnole-Santos, M. J.; Andrade, S. P. Angiotensin-(1-7): an update. Regulatory Peptides. 91:45-62, 2000).

The Mas regulator was initially described as a proto-oncogene due to its weak tumorigenic activity in vivo (Young D. Waitches G, Birchmeier C. Fasano O. Wigler M. Isolation and characterization of a new cellular oncogene encoding a protein with multiple potential transmembrane domains. Cell. 1986; 45:711-71).

In mammalians the expression of its gene was detected predominantly in testis and different areas of the brains, including the hippocampus and tonsils and less strongly but at a significant level in the kidneys and heard (Bunnemann B, Fuxe K, Metzger R, Mullins J, Jackson T R, Hanley M R, Ganten D. Autoradiographic localization of Mas proto-oncogene mRNA in adult rat brain using in situ hybridization. Neurosci Lett. 114:147-153,1990; Alenina N, Bader M, Walther T. Imprinting of the murine MAS proto-oncogene is restricted to its antisense RNA. Biochem Biophys Res Commun. 290:1072-1078, 2002).

Its gene modifies proteins with 7 transmembrane domains, which has characteristics of class I of G-protein-coupled receptors. In the initial studies, this gene was suggested as a codifier of a receptor for the octapeptide, angiotensin II (Jackson T R, Blair L A, Marshall J, Goedert M, Hanley M R. The Mas oncogene encodes an angiotensin receptor. Nature 335:437-440, 1988). However, Ambroz and co-workers and later Ardaillou showed that the elevation of intracellular calcium by Ang II in Mas-transfected cells only occurred in cells that expressed endogenously AT1 receptors for Ang II (Ambroz C, Clark A, Catt K J. The Mas oncogene enhances angiotensin-induced [Ca²⁺]i responses in cells with pre-existing angiotensin II receptors. Biochem Biophys Acta. 1133: 107-111, 1991; Ardaillou R. Angiotensin II receptors. J Am Soc Nephrol. 10:S30-S39, 1999). More recently it was observed that, in fact, the Mas in an receptor for Ang-(1-7) (Santos, R. A., Simōes e Silva A C, Maric, C., Silva, D. M., Machado, R. P., de Buhr, I., Heringer-Walther, S., Pinheiro, S. V., Lopes, M. T., Bader, M., Mendes, E. P., Lemos, V. S., Campagnole-Santos, M. J., Schultheiss, H. P., Speth, R.; Walther, T. Angiotensin-(1-7) is an endogenous ligand for the G protein-coupled receptor Mas Proc Natl. Acad Sci USA, 100 (14):8258-8263, 2003).

The endothelial dysfunction is an event that is more precocious at the installation and development of various pathologies related to the lesion of target-organs (heart, kidney, brain, blood vessels, reproductive organs, among others) (Goligorsky M S. Endothelial cell dysfunction: can't live with it, how to live without it. Am J. Physiol Renal Physilo. 288(5):F871-80, 2005). The reduction of the bioavailability of nitric oxide is a crucial factor for the beginning of endothelial dysfunction, since this molecule has vasodilative, antiproliferative, antithrombogenic, antiatherogenic properties and neutralizes the generation of reactive species of oxygen (Ogita H, Liao J. Endothelial function and oxidative stress. Endothelium. 11(2):123-32, 2004). Recently it was demonstrated that besides the classic pathway dependent on calcium, nitric oxide may be formed trough direct phosphorylation of sites, such as 1177 serine of the endothelial nitric oxide synthase (eNOS), through the B/Akt kinase protein. This mechanism contributes greatly to the maintenance of the endothelial integrity. The activation of Akt, by phosphorylating eNOS, participates in the nitric-oxide release stimulated by Ant-(1-7) in the human endothelium and, consequently, in the improvement of the endothelial functionality, a characteristic of the present invention. Another characteristic of the present invention is to demonstrate that Ang-(1-7) modulates negatively the actions of Ang II in the human endothelium, by inhibiting proximal intracellular pathways involved in the generation of reactive oxygen species, such as c-SRC. Additionally, Ang-(1-7) inhibits the activity of NAD(P)H oxidase, the largest source generating reactive oxygen species in the vascular wall (Touyz R M. Reactive oxygen species and angiotensin II signaling in vascular cells—implications in cardiovascular disease. Braz J Med Bio Res. 37(8): 1263-73, 2004). The activation of NAD(P)H by Ang II requires the presence of c-SRC for phosphorylation and migration to the membrane of the subunit p47phox of the enzyme. In addition, in the chronic stimulation with Ang II, the c-SRC participates in the increase of the protein expression of the subunits gp91phox, p22phox, and p47phox of NAD(P)H oxidase. (Touyz R M, Yao G, Schiffrin E L. c-Src induces phosphorylation and translocation of p47pho role in super oxide generation by angiotensin II in human vascular smooth muscles cells. Arterioscler Thromb Vasc Biol 23(6):981-7, 2003). This fact is particularly important, since the unbalance between pro- and antioxidant factors is one of the determinants of the start of endothelial dysfunction. Thus, besides contributing directly to the maintenance of the endothelial integrity by releasing the nitric oxide, Ang-(1-7) also neutralizes the generation of oxidizing free radicals. In turn, the generation of reactive oxygen species has a close relation with the apoptotic activity, since it stimulates signaling cascades, including MAPKs, caspases and others, determinants of cell death (Matuzawa, A., Ichijo, H. Stress-responsive protein kinases in redox-regulated apoptosis signaling. Antioxid Redos Signal 7(3-4):472-81; 2005). However, in the prior art there is no invention dealing with the use of formulations of Ang-(1-7) or its peptidic or non-peptidic analogs that activate, through Mas receptor, the cascade P13K/Akt/eNOS, as well as inactivate the NAD(P)H oxidase and reduce mechanisms that participate in the generation of reactive oxygen species and endothelial apoptosis, for prevention and treatment of pathologies that involve endothelial dysfunction, as for example, but not limited to cardiovascular, renal diseases, plurimetabolic syndrome, erectile dysfunction, disease of the central nervous system, among others.

Ant-(1-7) increases the exploratory behavior and facilitates memory (Santos, R A, Campagnole-Santos, M J. Central and Peripheral actions of Angiotensin-(1-7). Braz J Med Bio Res. 27(4):1033-47, 1994; Hellner, K., Walther, T., Schubert, M., Albrecht, D. Angiotensin-(1-7) enhances LTP in the hippocampus through the G-protein-coupled receptor Mas. Mol Cell Neurosci. 29 (3):427-35, 2005). The Mas receptor is located in cerebral areas involved in these functions, including the hippocampus, amygdale and brain-cortex (K. A Martin, S. G. Grant, S. Hockfield. The Mas proto-oncogene is developmentally regulated in the rat central nervous system. Brain Res Dev Brain Res 68(1):75-82, 1992). The interaction of the Mas receptor with Ang-(1-7) activates signaling pathways with anti-apoptotic characteristics, more specifically the pathway of the PKI3/Akt. Phosphorylation of the B (Akt) kinase protein, which is increased by the Ang-(1-7), reduces cellular apoptosis (Z.-Z. Yang, O. Tschopp, A . Baudry, B. Dümmler, D. Hynx and B. A Hemmings. Physiological functions of protein kinase B Akt Biochem Soc Trans. 32:350-354, 2004). Knockout mice for Akt γ, the cerebral sub form of Akt, exhibit a dramatic reduction of the cerebral weight (Z.-Z. Yang, O. Tschopp, A. Baudry, B. Dümmler, D. Hynx and B. A Hemmings. Physiological functions of protein kinase B Akt Biochem Soc Trans. 32:350-354, 2004). Some of the sites of expression of the Akt in the brain are the hippocampus and the cerebral cortex, regions also rich in RNAM for the Mas receptor. However, in the prior art there is no invention dealing with formulations of angiotensin-(1-7) for controlling or preventing degenerative brain diseases or memory or learning disorders, based on the stimulation of the anti-apoptotic activity mediated by Akt, induced by interaction of Ang-(1-7) with the Mas receptor. Similarly, there is no invention dealing with the use of formulations of Mas receptor agonists or antagonists for studies, prevention or treatment of degenerative brain diseases or memory or learning disorders, based on the stimulation of the anti-apoptotic activity mediated by Akt, induced by interaction of peptidic or non-peptidic Mas receptor agonists with this receptor.

The present invention is characterized by the use of controlled-release systems containing Ang-(1-7), analogs or derivatives of Ang-(1-7), which facilitate the access for interaction with the Mas G-protein-coupled receptor. This interaction between the G protein, Mas and Ang-(1-7), the analogs or derivatives, enables the control or preventions of degenerative brain diseases characterized by an increase in the apoptotic activity, including degenerative brain disorders such as Alzheimer, Parkinson, Huntington diseases, among others. The satisfactory controlled-release systems include but are not limited to cyclodextrines, biocompatible polymers, biodegradable polymers, other polymeric matrices, capsules, microcapsules, microparticles, preparations of bolus, osmotic pumps, diffusion devices, liposomes, lipospheres, and transdermic administrative systems.

Recently, various papers showed that the IP3k/AKT pathway plays a critical role for mediating the insulin receptor signaling with its substrates (Zdychova J, Komers R. Emerging role of Akt kinase/protein kinase B signaling in pathophysiology of diabetes and its complications. Physiol Res; 54(1):1-16, 2005). Mediators that alter this cascade, as growth factors, angiotensin II, reactive oxygen species, corticosteroids, estrogen and the alteration itself of the glycemic state will promote proliferative cellular alterations that will lead, since the PI3K/Akt pathway is an essentially anti-apoptotic pathway, from endothelial dysfunction inherent in diabetes to embryonal teratogenic alterations in diabetic pregnant women (Reece E A, Ma X D, Zhao Z, Wu Y K, Dhanasekaran D. Aberrant patterns of cellular communication in diabetes-induced embryopathy in rats: II, apoptotic pathways Am J. Obstet Gynecol. 192(3):967-972, 2005). The Akt may be regulated by various factors that direct the signaling mediated by this pathway. For example, D-glucose regulates the phosphorylation of Akt, and hyperglycemy has been related to endothelial dysfunction in diabetes (Varma S, Lal B K, Zhen R, Breslin J W, Saito S, Pappas P J, Hobson Ii R W, Duran W N. Hyperglycemia Alters PI3k and Akt Signaling and Leads to Endothelial Cell Proliferative Dysfunction. Am J Physiol Heart Circ Physiol. 2005 in press). In addition, phosphorylation of serine is related to the activation of e-NOS (Kobayashi T, Taguchi K, Yasuhiro T, Matsumoto T, Kamata K. Impairment of PI3-K/Akt pathway underlies attenuated endothelial function in aorta of type 2 diabetic mouse model. Hypertension. 44(6):956-962, 2004) and can be inhibited by increasing lipidic levels, suggesting that, beside endothelial preservation and activation of the circulating endothelial progenitory cells (EPCs), Akt may be related to atheroprotective effect. In the skeletal muscle, alterations in the phosphorylation of Akt is related to modification in the traffic via GLUt4 in type 2 diabetes (Karlsson H K, Zierath J R, Kane S, Krook A, Lienhard G E, Wallberg-Henriksson H. Insulin-Stimulated Phosphorylation of the Akt Substrate AS160 Is Impaired in Skeletal Muscle of Type 2 Diabetic Subjects. Diabetes. 54(6):1692-7, 2005). Another interesting aspect is that this cascade seems to be involved in the proliferation and survival of the β cells themselves and that its inactivation by ceramide activated phosphatases (CAPP) might cause alterations in the secretion of insulin in the type I diabetes (Kowluru A. Novel regulatory roles for protein phosphatase-2A in the islet beta cell. Biochem Pharmacol. 69(12): 1681-1691, 2005). The existence of a negative feedback process mediated by the PI3K/Akt/TOR pathway was pointed out as a critical event for the resistance to the insulin and tumorigenesis. (Manning B D, Balancing Akt with S6K: implications for both metabolic diseases and tumorigenesis, J. Cell Biol, 167(3):399-403, 2004). Thus, the participation of the Akt pathway both in the causing factors and in the complications resulting from diabetes, such as the vasculopathies, is evident.

There seems to be a close relationship between the rennin-angiotensin system and the insulin receptor signaling. It has already been demonstrated that Ang II inhibits the phosphorylation of Akt mediated by the insulin receptor. In addition, the oxidative stress stimulated by Ang II also alters various steps of the intracellular cascade activated by insulin (Taniyama Y, Hitomi H, Shah A, Alexander R W, Griendling K K. Mechanisms of reactive oxygen species-dependent downregulation of insulin receptor substrate-1 by angiotensin II. Arterioscler Thromb Vasc. Biol. 25 (6):1142-1147, 2005). This explains, in part, why the use of Ang II inhibitors improves the resistance to insulin and, consequently, co-morbidity associated to diabetes, like micro-vascular lesion. Ang-(1-7) is a potent biological Ang II antagonist and has various actions related with the improvement of the endothelial function. Its levels are raised during the pharmacological blocking of the system, indicating that Ang-(1-7) is an important mediator of the beneficial effects of both ACE inhibitors and AT1 receptor antagonists. As already mentioned above, the attachment of Ang-(1-7) to the Mas receptor leads to the strong phosphorylation of Akt. However, in the prior art there is no invention dealing with the use of formulations of angiotensin-(1-7) or its peptidic or non-peptidic analogs for study, prevention or treatment consequent to the resistance to insulin or deficiency of production of this hormone.

Angiotensin-(1-7) is present in the heart and has important cardiac effects such as increase in the contractility and reduction of cardiac arrhythmias (Ferreira, A J and Santos, R A S. Cardiovascular actions of Angiotensin-(1-7). Braz. J. Med. Biol Res. 38(4):499-507, 2005). The Mas receptor is also expressed in the heart and the deficiency thereof entails an important reduction of the cardiac function (Ferreira, A J Santos, R A S. Cardiovascular actions of Angiotensin-(1-7), Braz. J. Med. Biol Res. 38(4):499-507, 2005). The kinase Akt protein is also expressed in the heart, especially in cardiomyocytes. In these cells Akt is also phosphorylated via PIK3, increasing the myocardial contractility and reducing reperfusion arrhythmias. Mice with increased Akt expression in the heart exhibit alterations in the synthesis of proteins involved in the glycolytic pathway, like an increase in the “insulin-like growth factor-binding protein 5”, which ends up raising the activity of this path way (Latronico, M G V, Costinean, S., Lavitrano, M. L. Peschle, C., Condorelli, G. Regulation of Cell Size and Contractile Function by AKT in Cardiomyocytes, Ann. N.Y. Acad. Sci. 1015: 250-260, 2004; Cook, S. A. Matsui, T., Li, L., Rosenzweig, A . Transcriptional Effects of Chronic Akt Activation in the Heart. J Biol Chem: 277(25): 22528-22533, 2002). The administration of Ang-(1-7) protects the heart of the consequences against myocardial infarct (Loot A. E., Roks A . J., HENNING, R. H., Tio, R. A. , Suurmeijer, A. J., Boomsma, F, van Gilst, W. H. Angiotensin-(1-7) attenuates the development of heart failure after myocardial infarction in rats. Circulation. 2002:105 (13):1548-50). Transgenic rats that expresses an Ang-(107) producing fusion protein have lower cardiac hypertrophy in response to treatment with isoproterenol and shorter duration and occurrence of reperfusion arrhythmias (Santos, R. A., Ferreira, A. J., Nadu, A. P., Braga, A. N., de A.meida, A. P., Campagnole-Santos, M. J., Baltatu. O., Iliescu, R., Reudelhuber, T. L., Bader, M. Expression of an angiotensin-(1-7)-producing fusion protein produces cardioprotective effects in rats. Physiol Genomics. 2004; 19(7):292-9). On the other hand, intracoronary administration of Akt gene via adenovirus, produced reduction of the size of the infarted area in rats (W. Miao, Z. Luo, R. N. Kitsis and K. Walsh. Intracoronary, Adenovirus-mediated Akt Gene Transfer in Heart Limits Infarct Sice Following Ischemia-reperfusion Injury in vivo. J Mol Cell Cardiol. 32:2397-2402, 2000). Stem cells modified with Akt prevent the remodeling and restore the cardiac function of infarcted hearts in rats. (Mangi, A. A, Noiseux, N., Kong, D., He, H., rezvani, M., Ingwall, J. S., Dzau, V. J. Mesenchymal stem cells modified with Akt prevent remodeling and restore performance of infarcted hearts Nat Med. 9:1195-1201, 2003). However, in the prior art there is no invention dealing with the use of formulations of angiotensin-(1-7) or of its peptidic or non-peptidic analogs, for increasing heart performance or control or prevention of myocardial degenerative diseases, increase of the viability of stem cells after intracardiac administration, or reduction of cardiac remodeling or electrophysiological disorders of the heart based on the stimulation of intracellular transduction pathways like that of anti-apoptotic activity produced by stimulation of the PIK3/Akt pathway, by interaction with Ang-(1-7) with the Mas receptor.

Muscular atrophy is a serious morbidity caused by a variety of conditions such as cachexia, cancer, AIDS, prolonged restriction to bed due to numberless factors, diabetes, chronic use of corticoids and varied neurological syndromes and traumatisms (Lai K M, Gonzalez M. Poueymirou W T, Kline W O, Na E, Zlotchenko E, Stitt tN, Ecomonides An, Yancopoulos G D, Glass D J. Conditional activation of act in adult skeletal muscle induces rapid hypertrophy. Mol Cell Biol. (21):9295-304, 2004). Recently, strategies that can activate signaling pathways in the skeletal muscle capable of restoring the muscular tropism have been studied. Among these pathways, Akt deserves to be highlighted because it is capable of activating anabolic pathways and is simultaneous and predominantly capable of suppressing catabolic pathways (Stitt T N, Duran D., Clarke B A, Planar F, Timofeyva Y, Kline W O, Gonzalez M, Yancopoulos G D, Glass D J. Mol Cell. 14(3):395-403, 2004). The IGF-1/PI3K/Akt pathway prevents expression of muscle atrophy induced ubiquitin ligases by inhibiting FOXO transcription factors. Mol Cell; 14(3):395-403, 2004). The actions of Ang-(1-7) in the peripheral musculature include increase in the blood flow in the skeletal muscle (Sampaio W O, Nascimento A A S, Santos R A S, Systemic and regional hemodynamic effects of angiotensin-(1-7) in rats. Am J Physiol Heart Circ Physiol., 284(6):H1985-94, 2003) and synaptic facilitation (Bevilaqua E R, Kushmerick C, Beirão P S, Naves L A. Angiotensin 1-7 increases quantal content and facilitation at the frog neuromuscular junction. Brain Res.; 927(2)208-11, 2002). In addition Ang-(1-7) activates Akt. However, in the prior art there is not invention dealing with the use of formulations of Ang-(1-7) or peptidic or non-peptidic analogs thereof, which activate the Ang-(1-7)/Mas/Akt axis for prevention and treatment of pathologies that involve alterations in the differentiation, maturation and regeneration of muscle, as well as for use as an ergogenic resource.

The activation of the anti-apoptotic activity based on the activation of Akt mediated by interaction of Ang-(1-7) or other agonists with the Mas receptor may also occur in other tissues and organs, including, among others, the skin, endocrinal glands, liver, kidney, gastrointestinal tract and genitourinary tract.

The present invention can be better understood with the aid of the following examples and detailed description, which are not limitative.

### EXAMPLE 1

This example describes the identification of the MAS receptor in cerebral areas involved in the central control of physiological functions.

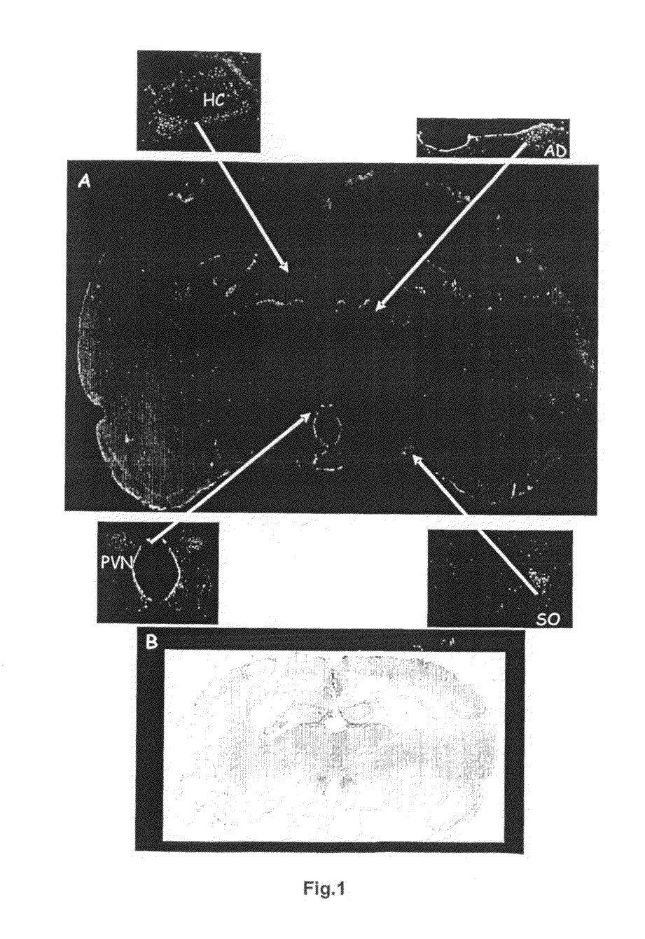

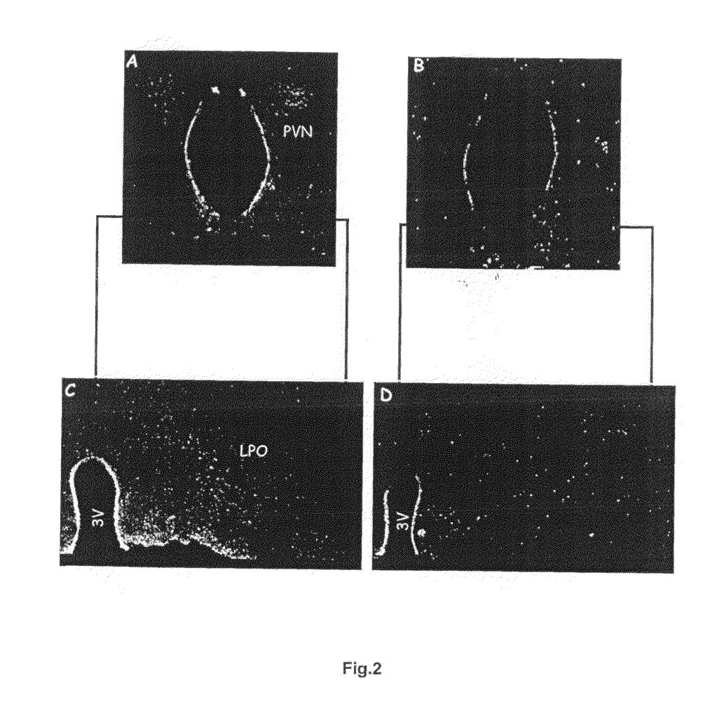

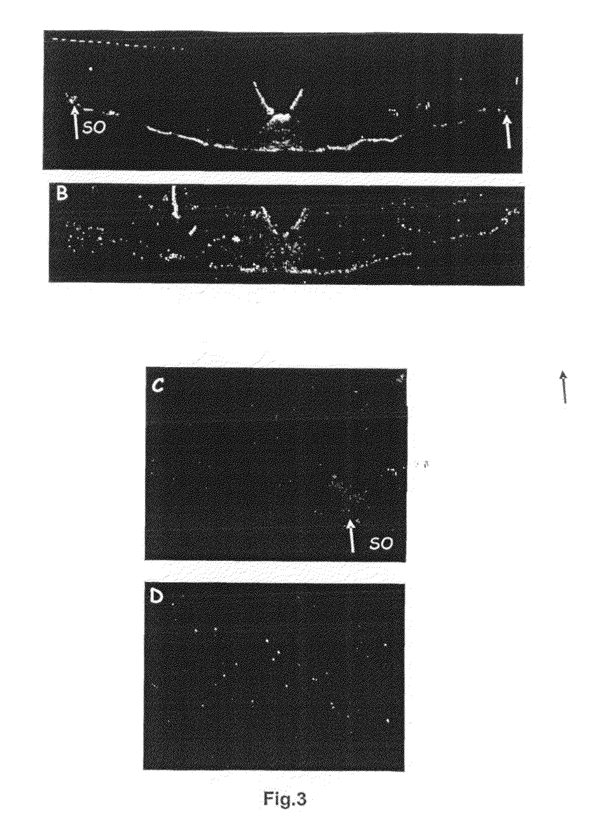

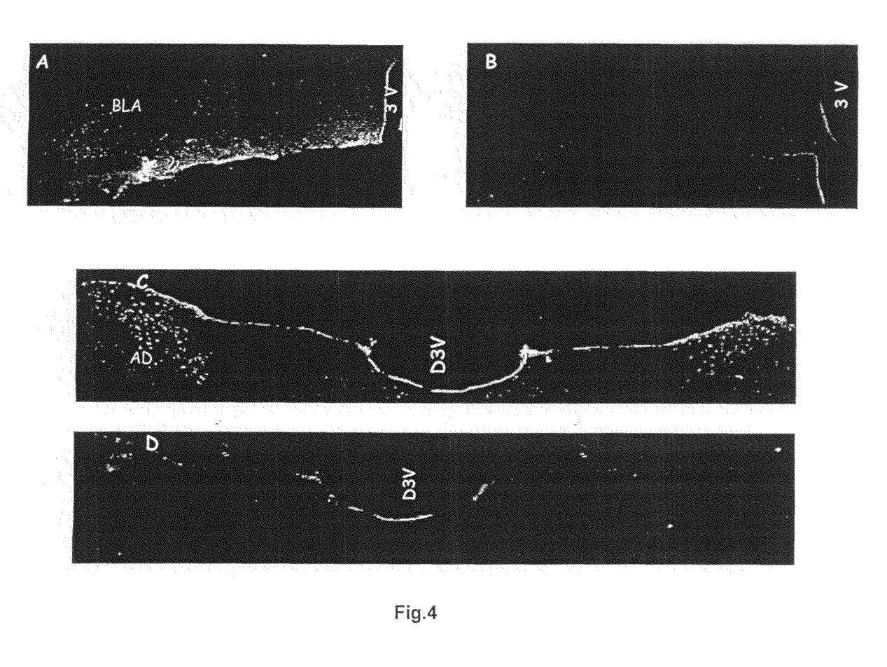

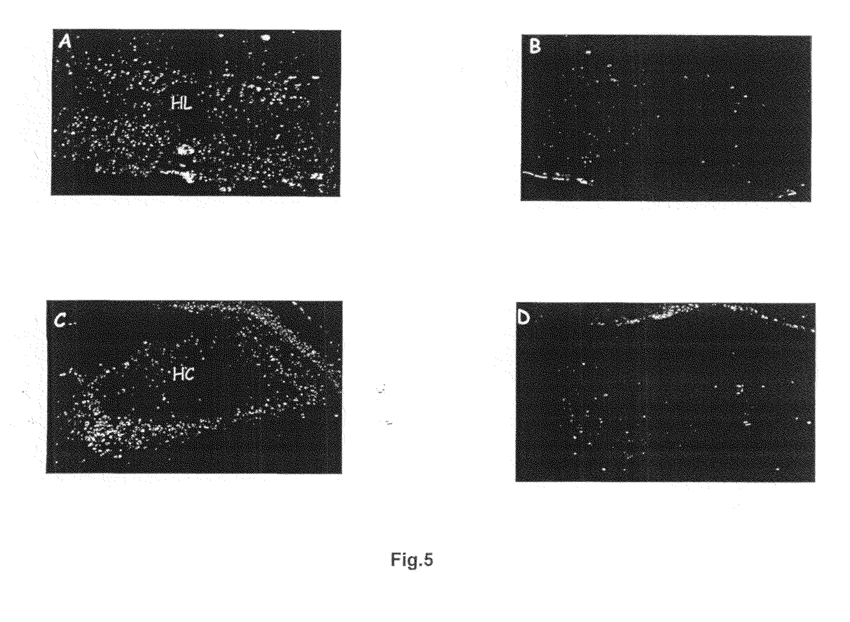

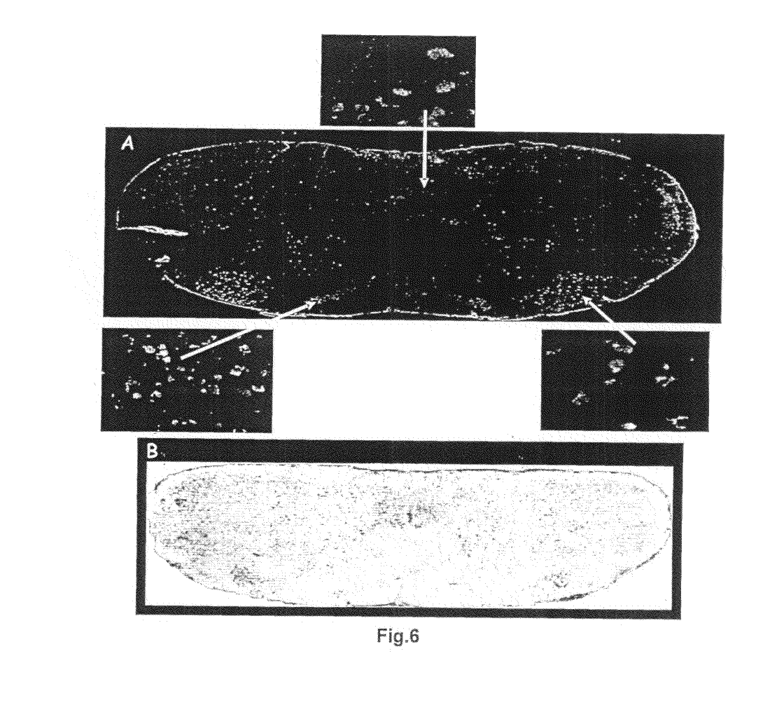

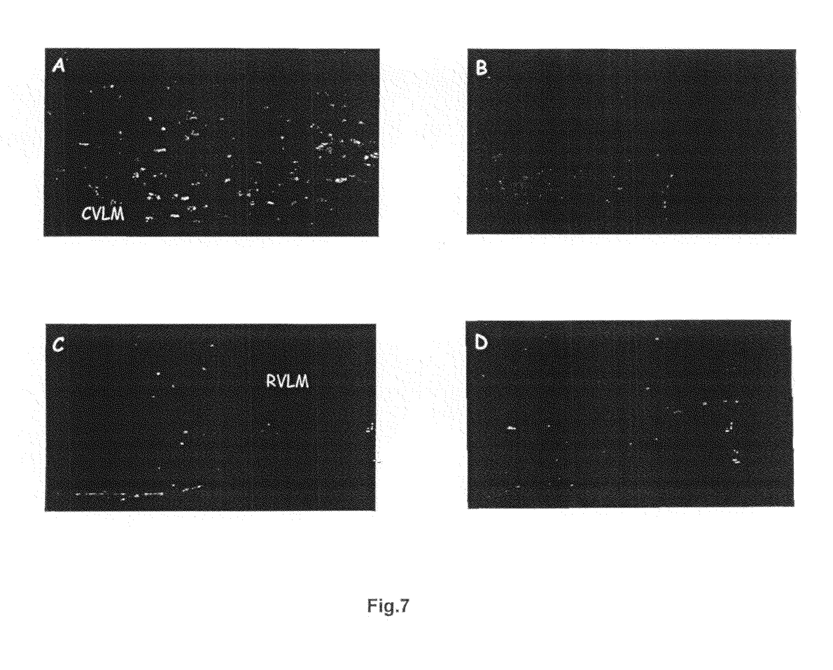

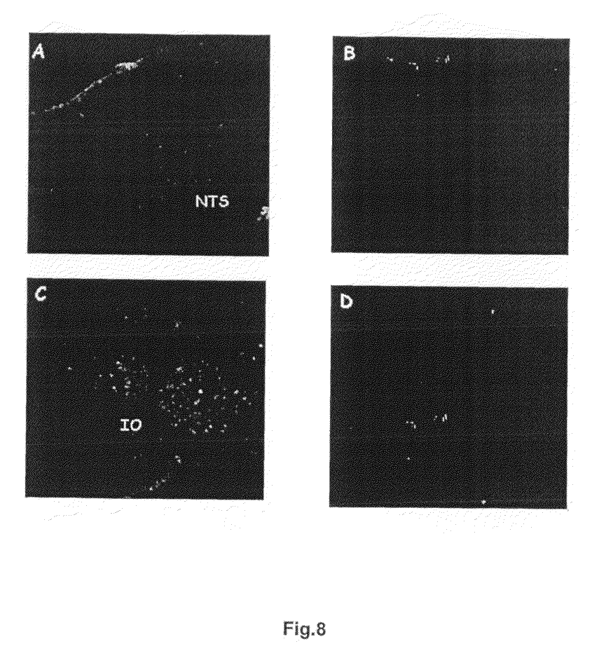

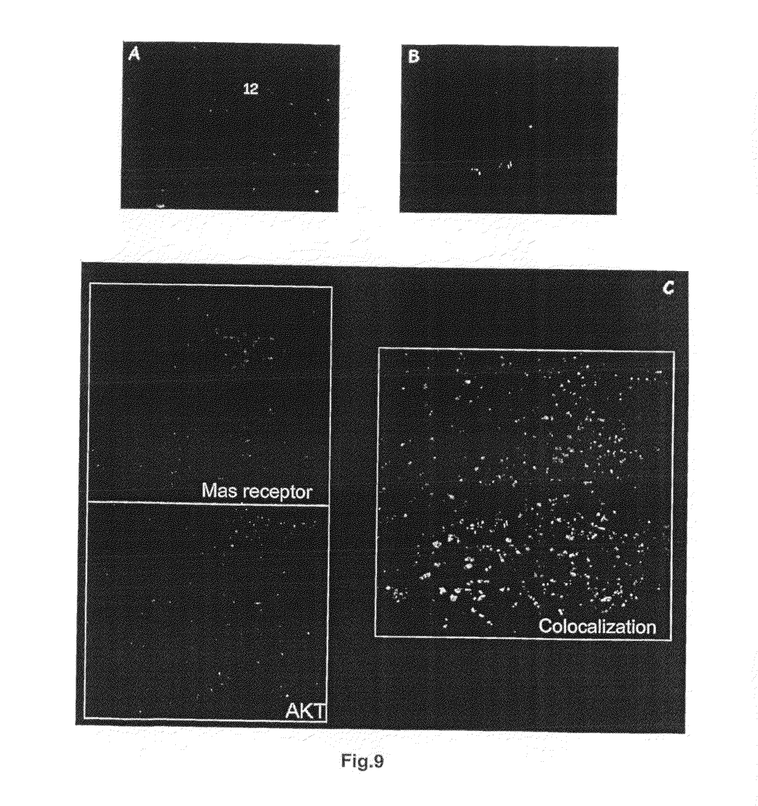

The animals were anesthetized with tribromoethanol (0.25 g/Kg), and then transcardially perfused for 2 minutes with PBS (0.02 M pH 7.4), then for 15 minutes with a 10% paraformaldehyde solution in PBS. The brain was withdrawn and placed into the same fixing solution for 2 h. Then the tissue was washed 3 times in PBS solution and afterwards placed into a sucrose solution (30% in PBS) overnight. Cuts of 30 μm of the brain were made in the frontal plane in freezing microtome at the temperature of −18° C. Cuts of the bulb and of the hypothalamus were incubated by the “free floating” method in PBS, tween 0.5% and BSA 5% for 15 minutes each, then the cuts were incubated with Mas primary antibody (1:500) for 48 hours at 4° C. The negative control was carried out in adjacent cuts incubated with primary antibody pre-absorbed by the Mas protein. After 48 hours the cuts were 3 times for 5 minutes in PBS solution and then incubated with the secondary conjugated antibody with fluorescent compounds for 60 minutes at room temperature. After this period the cuts were washed 3 times for 5 minutes in PBS and kept in dry gelatinized slides and covered with glass slides in mounting solution containing 1:3 glycerol and PBS, respectively. The blades were analyzed under a confocal microscope with specific exciting and emitting filters for each fluorescent compound used. Slides containing adjacent cuts subjected to immunofluorescence assay were stained by the neutral red method for structural analysis of he tissue and identification of the different areas. One used the Atlas de G. Paxinos, C. Watson, The rat brain in stereotaxic coordinates, 2^{nd} Edition, Academic Press, New York, 1986, for defining the areas observed in the brain. FIG. 1 shows, in a frontal cut of the hypothalamus, the presence by immunoreactivity of the Mas Ang-(1-7) receptor, in a number of areas (FIG. 1A) and in adjacent cut stained with neutral red for the histological identification of the different areas (FIG. 1B). FIG. 2 shows the presence by immunoreactivity of the Mas Ang(1-7) receptor, in the paraventricular nucleus (PVN, FIG. 2A) and lateral pre-optic area (LPO, FIG. 2C) and, in adjacent cuts (FIGS. 2B and 2D), the controls, showing the disappearance of the marking when pre-absorption of the antibody by the synthetic Mas protein is carried out. In FIG. 3, the arrows show the presence of the Mas, Ang-(1-7) receptor, by immunoreactivity, in the supra-optic nucleus (CSO, FIG. 3A). Adjacent cuts (FIGS. 3B and 3D) showing the disappearance of the marking when pre-absorption of the antibody by the synthetic Mas protein is carried out. FIG. 4 shows the presence of the Mas Ang-(1-7) receptor, by immunoreactivity, in the tonsils (FIG. 4A) and anterodorsal nucleus of the thalamus (FIG. 4C) and the controls in adjacent cuts (FIGS. 4B and 4D) showing the disappearance of the marking when pre-absorption of the antibody by the Mas synthetic protein is carried out. FIG. 5 shows the presence of the Mas Ang-(1-7) receptor, by immunoreactivity, in the cortex (HL, FIG. 5A) and hippocampus (HC, FIG. 5C) and its controls in adjacent cuts (FIGS. 5B and 5D) showing the disappearance of the marking when the pre-absorption of the antibody by the Mas synthetic protein is carried out. FIG. 6 shows in A a frontal cut of the bulb illustrating the immunoreactivity for the Mas Ang-(1-7) receptor in a number of areas. In B and in adjacent cuts, stained with neutral red for histological identification of the different areas. FIG. 7 shows the immunoreactivity for the Mas Ang-(1-7) receptor in the caudal ventrolateral area (CVLM, FIG. 7A) and rostral ventrolateral area of the bulb (RVLM), FIG. 7C) and its controls in adjacent cuts (FIGS. 7B and 7D) showing the disappearance of the marking when pre-absorption of the antibody by the Mas synthetic protein is carried out. FIG. 8 shows the presence of the Mas Ang-(1-7) receptor, by immunoreactivity, in the nucleus of the solitary tract (NTS, FIG. 8A) and in inferior olive nucleus (IO, FIG. 8C) and its controls in adjacent cuts (FIGS. 8B and 8D) showing the disappearance of the marking when pre-absorption of the antibody by the Mas synthetic protein is effected. FIG. 9 shows the presence of the Mas Ang-(1-7) receptor, by immunoreactivity, in the hypoglossus (12, FIG. 9A) and its control in adjacent cut (FIG. 9B) showing the disappearance of the marking when pre-absorption of the antibody by the Mas synthetic protein is carried out. FIG. 9C shows the immunocolocalization of the Mas receptor and of the AKT in the rostral ventrolateral area of the bulb, indicating a possible interaction between the receptor Mas and the AKT in the neural modulation of this area.

### EXAMPLE 2

This example describes the identification of the activation of the PIK3/Akt pathway by interaction of Ang-(1-7) with its Mas receptor.

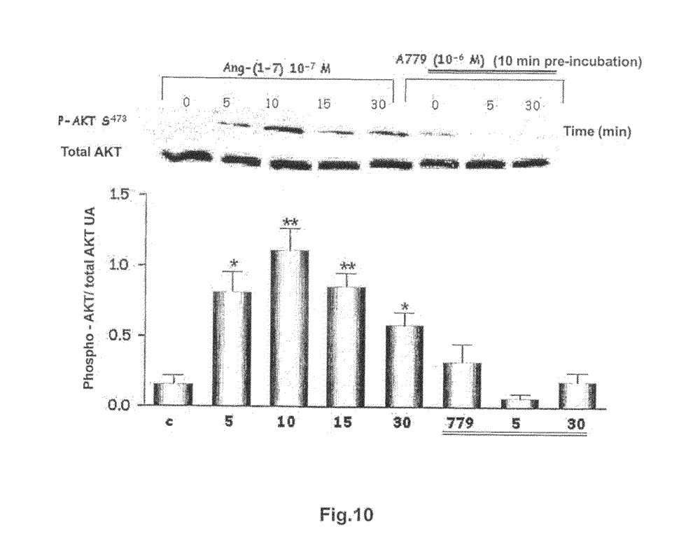

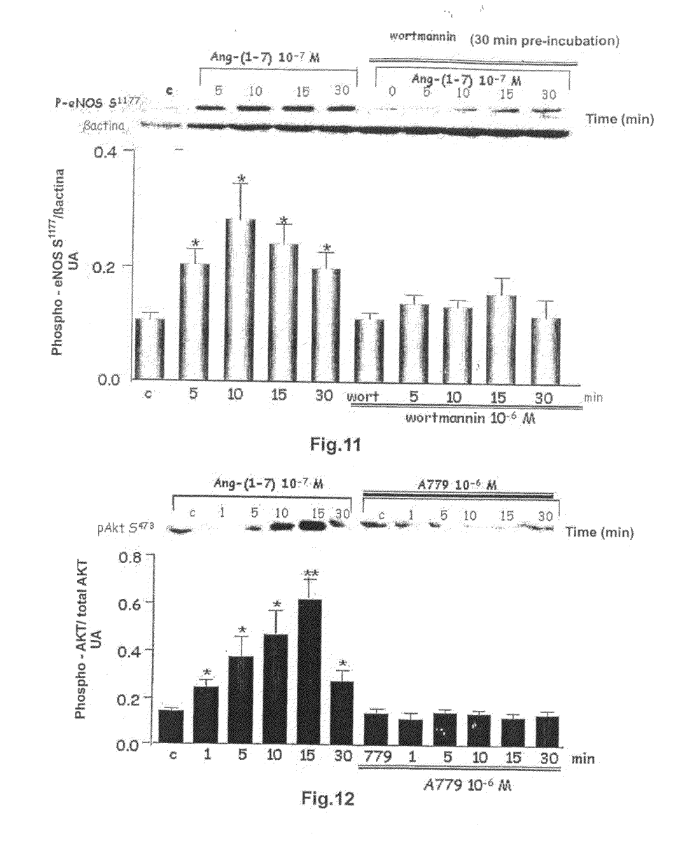

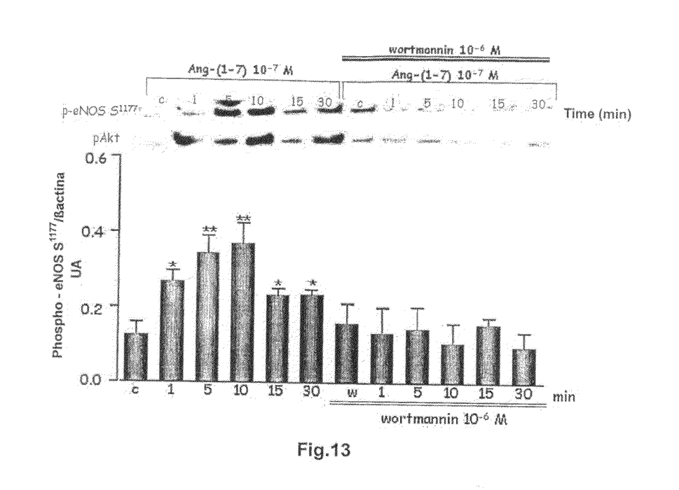

CHO cells transfected with the Mas receptor (CHO-Mas) and human endothelial cells of the thoracic aorta (HAEC) were cultured until confluence of approximately 80% and processed with a lise buffer for Western blotting. After the processing, the protein concentration was determined and the lisates were subjected to electrophoresis in polyacrylamide/SDS gel and then subjected to transfer to the nitrocellulose membrane. The membranes were incubated with specific antibodies (anti-phospho-Akt, anti-Akt, anti-phospho-eNOS and anti-β-actin). The bands were viewed after development by chemoluminescence. FIG. 10 shows the stimulation produced by Ang-(1-7) in the phosphorylation of kinase B (Akt) in CHO-Mas cells. The Ang-(1-7) antagonist, A-779 blocked this effect. FIG. 11 shows that Akt participates in the phosphorylation of the stimulatory site of the endothelial nitric oxide synthase (S1177) stimulated by Ang-(1-7) in the CHO-Mas cells. The phosphatidylinositol 3 kinase antagonist (PI3K) blocked this effect. FIG. 12 shows the stimulatory effect of Ant-(1-7) in the phosphorylation of kinase B (Akt) on the human endothelial cells (HAEC). The Ang-(1-7) antagonist, A-779 blocked this effect. FIG. 13 shows the participation of Akt in the phosphorylation of the stimulatory site of eNOS (S1177) stimulated by Ang-(1-7) in the human endothelial cells (HAEC). The PI3K antagonist, wortmannin, blocked this effect.

### EXAMPLE 3

This example describes the identification of the participation of the PIK3/Akt pathway in the improvement of the endothelial function stimulated by An-(1-7), via Mas receptor, in awake rats.

Wistar rats were subjected, 24 hours before the experiments, to surgical implantation of catheters into the femoral artery (for analysis of blood pressure and heart rate), femoral vein (for injection and infusion of drugs) and left carotid artery (for injection of drugs). The records of blood pressure and heart rate were obtained through a data acquisition system connected to a microcomputer (BIOPAC System, INc.). FIG. 14 A shows that the vasodilating action of acetylcholine (ACh) is not altered by endovenous infusion of saline (NaCl 0.9%, 0.4 mL/h), in awake Wistar rats (n=7). However, the endovenous infusion of Ang-(1-7) (7.0 pmol/min) potentiates the vasodilating action of acetylcholine (ACh) in awake Wistar rats (n=9) (FIG. 14B). FIG. 14.C shows that in bolus endovenous injection of wortmannin (10⁻⁶M), PI3k inhibitor, followed by endovenous infusion of wortmannin (10⁻⁶M) associated to Ang-(1-7) (7.0 pmol/min), blocks the potentiation of Ang-(1-7) on the vasodilating effect of acetylcholine (ACh) in awake Wister rats (n=7). In none of the groups did we observe any alterations in the baroreflex.

### EXAMPLE 4

This example describes the identification of the activation of the PIK3/Akt pathway by interaction of Ang-(1-7) with the Mas receptor in the activity of the NADPH oxidase.

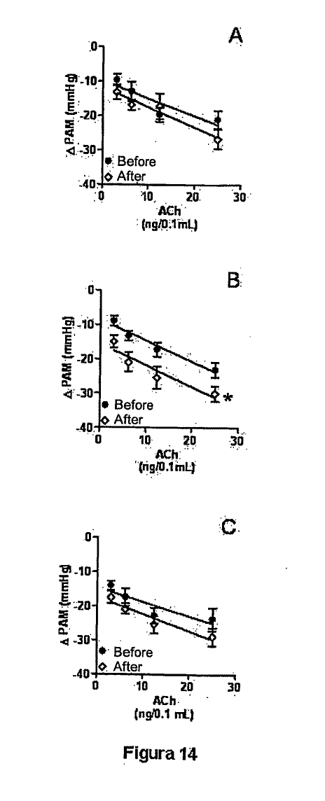

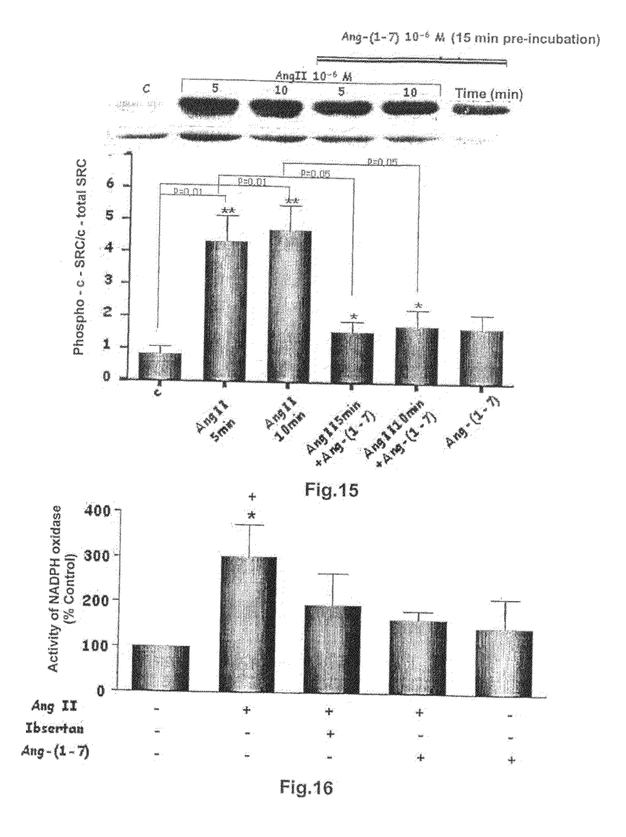

In order to quantify the activity of the NAD(P)H Oxtidase, human aorta endothelial cells (HAEC) were stimulated with angiotensin II (10⁻⁷M) for 10 minutes. In some experiments, the cells were pre-exposed to the AT₁ receptor antagonist Ibesartan (10⁻⁵M), for 30 minutes or to Ang-(1-7) (10⁻⁷ M) for 15 minutes. The chemoluminescence derived from lucigenin was used to determine the activity of NAD(P)H oxidase in the homogenate of the cells. In order to quantify the modulation of Ang-(1-7) in the phosphorylation of c-SRC, the HAECs were cultured until confluence of about 80% was reached and processed with lise buffer for Western blotting. After the processing, the protein concentration was determined and the lisates were subjected to gel electroforesis of polyacrylamide/SDS and then to the transfer to nitrocellulose membrane. The membranes were incubated with specific antibodies (anti-phospho-c-SRC, anti-c-SRC). The bands were visualized after development by chemoluminescency. FIG. 15 shows the modulating effect of Ang-(1-7) in the phosphorylation of c-SRC stimulated by Ang II in the human endothelial cells (HAEC). The bar graph shows the average±EPM of 4 experiments. *P<0.05 and **P<0.001 vs control. FIG. 16 shows the effect of Ang-(1-7) (10⁻⁷ M, 15 min of pre-incubation) on the activity of NAD(P)H oxidase in HAEC stimulated by Ang II (10⁻⁷ M, 10 min). In some experiments the cells were pre-incubated with Ibesartan (10⁻⁵ M, 30 min). Data are presented with an average±EPM of 4 experiments. *p<0.05 vs control.+p<0.05 vs Ang II+Ang (1-7).

### EXAMPLE 5

This example describes the effect of the Mas, G-protein-coupled receptor antagonist in the spermatogenesis.

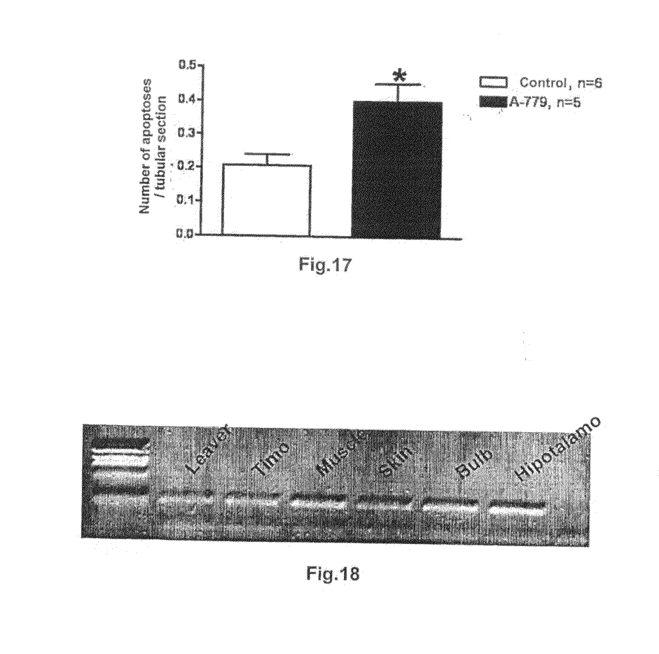

Osmotic mini-pumps (ALZET, model 2002) containing the Mas G-protein-coupled receptor antagonist, A-779 (2.5 μg/h, 14 days, n=5) or carrier (NaCl 0.9%, 1 μl/h, 14 days, n=6) were implanted subscutaneously into the dorsal region of C57 mice under anesthesia with tribromoethanol (2.5%, 1 ml/100 g of body weight). After this period, the animals were weigh and then injected, by intraperitoneal route, with heparin at the concentration of 125 Ul/kg of body weight. After fifteen minutes, these animals were sedated with sodium thiopental (50 mg/kg of body weight) and perfused through the left ventricle. In a first step, it was carried out the washing of the vascular bed with a 0.9% saline solution, under a pressure of approximately 80 mmHg, for about 5 minutes, at room temperature. Immediately after this procedure, the animals were perfused with a 4% glutaraldehyde fixing solution in a phosphate buffer (0.05M, pH 7.2-7-4) for about 25 minutes. After this step, the testicles were removed and separated from the respective epididymis and weighed. From the testicular and body weights, one estimated the gonadosomatic index (percentage relation between the testicular weight and the body weight) for each animal. For the microscopic analyses, fragments of the testis up to about 3 mm thick were collected, which were dipped into glutaraldehyde buffered at 4% for two to four hours, at 4° C. Then, the fragments were stored in a phosphate buffer at 4° C., until they were processed for histological analysis (presence of apoptosis). These fragments of testicles were dehydrated at increasing concentrations of alcohol (70°, 80°, 90°, 100° with exchanges every thirty minutes. After dehydration, the fragments were included in metacrylate glycol (Leica Historesin Embeddding Kit, Leica Instruments), being subsequently sectioned in the thickness of 4 μm in a microtome with glass razor blades. The obtained sections were stained with 1% sodium toluidine-borate blue, mounted with Entellan (Merck), and analyzed under an Olympus microscope. FIG. 17 shows that the animals treated with the Mas G-protein-coupled receptor antagonist, A-779, exhibited a larger number of apoptosis per transverse section with respect to the control group, but there was not different in the gonadosomatic index.

### EXAMPLE 6

This example describes the morphologic alterations in ovaries of Mas-KO mice.

Immature female mice (30 days, 11-14 g) WT (wildi-type; n=4) and Mas-KO (n=3) were sacrificed by beheading. The ovaries were removed, fixed in 4% glutaraldehyde in PBS 0.1 m and included in glycolmetacrylate. Each 5^{th} cut (5 μm) was collected on a histological slide and stained with toluidine blue. Morphologic analyses were used for establishing the number of growing, antral and atresic primary follicles. The ovaries of KO exhibited a significantly lower total number of follicles than those of the WT (1494 vs 3332), especially of primordial follicles (418 vs 1930). In addition, the percentage of atresic follicles of the KO female mice was of about 50% higher when compared with that of the WT mice. These data indicate an increase in the ovarian apoptotic activity as a result of the deletion of the Mas receptor.

### EXAMPLE 7

This example demonstrates the expression of the Mas receptor, trough the RT-PCR, in a number of tissues where the presence of these receptors may contribute in the modulation of the apoptotic activity (FIG. 18).

1. Use of Mas G-protein-coupled receptor antagonists and agonists, characterized by modulation of the apoptotic activity involving alterations in the activity of the protein kinase B/Akt.

2. Use of Mas G-protein-coupled receptor antagonists and agonists, characterized by modulation of the production of oxygen reactive species.

3. Use of Mas G-protein-coupled receptor antagonists and agonists, characterized by the use of the Angiotensin-(1-7) peptide and its analogues, agonists and antagonists, either peptidic or non-peptidic, as modulators of the apoptotic activity in the prevention and treatment of diseases.

4. Use of Mas G-protein-coupled receptor antagonists and agonists, characterized by the use of the Angiotensin-(1-7) peptide and its analogues, agonists and antagonists, either peptidic or non-peptidic, as modulators of the apoptotic activity for use in the prevention or treatment of organic alterations produced by aging.

5. Use of Mas G-protein-coupled receptor antagonists and agonists, characterized by the use of formulations with pharmaceutically and pharmacologically acceptable excipients or carriers, of Mas G-protein-coupled receptor agonists and antagonists, including the peptide Angiotensin-(1-7) and its analogues, agonists and antagonists, either peptidic or non-peptidic, as modulators of the apoptotic activity.

6. Use of Mas G-protein-coupled receptor antagonists and agonists according to claim 1, characterized by the use of at least one further pharmacologically active compound and/or pharmaceutically acceptable carriers and/or excipients, including water, saline solution, buffered solutions, Ringer solution, dextrose solution, Hank solution, biocompatible saline solutions, either containing or not containing polyethylene glycol, nonaqueous vehicles, fixed oils such as sesame oil, ethyloleate, or triglyceride, sodium carboxymethylcellulose, sorbitol, or dextran, timerosal, morocresol, formalin and benzyl alcohol human-serum albumin, cyclodextrin, liposomes, cyclic or non-cyclic oligosaccharides.

7. Use of Mas G-protein-coupled receptor antagonists and agonists according to claim 1, characterized by the use of implantable or injectable micro and nanoparticular devices of formulations of the Mas G-protein receptor agonists or antagonists, including the Angiotensin-(1-7) peptide and its analogues, agonists and antagonists, either peptidic or non-peptidic, as modulators of the apoptotic activity.

8. Use of Mas G-protein-coupled receptor antagonists and agonists according to claim 7, characterized by the use of biodegradable polymers such as PLGA, PLA, PGA, caprolactone, combination of these polymers and liposomes.

9. Use of Mas G-protein-coupled receptor antagonists and agonists according to claim 1, characterized by the use of Mas G-protein-coupled receptor antagonists and agonists including the Angiotensin-(1-7) peptide and its analogues, agonists and antagonists, either peptidic or non-peptidic, as modulators of the apoptotic activity in the prevention and treatment of diseases that involve alterations in the muscular differentiation, maturation and regeneration in muscular atrophies such as cachexia, cancer, AIDS, prolonged restriction to bed due to numberless factors, diabetes, chronic use of corticoids and varied neurological syndromes, traumatisms and degenerative diseases that lead to muscular atrophy, as well as for the prevention or treatment of organic alterations produced by aging and as ergogenic aid.

10. Use of Mas G-protein-coupled receptor antagonists and agonists according to claim 1, characterized by the use of cDNAs that encode peptide sequences corresponding to Angiotensin-(1-7) and its analogues, agonists and antagonists, as apoptotic-activity modulating agents, for use in gene therapy of diseases, as well as of organic alterations produced by aging.

11. Use of Mas G-protein-coupled receptor antagonists and agonists according to claim 1, for use in gene therapy of diseases as well as organic alterations produced by aging, characterized by the use of Mas G-protein-coupled receptor antagonists and agonists, including the peptide Angiotensin-(1-7) and its analogues, agonists and antagonists, either peptidic or non-peptidic, as modulators of the apoptotic activity.

12. Use of Mas G-protein-coupled receptor antagonists and agonists according to claim 1, characterized by the use of Mas G-protein-coupled receptor antagonists and agonists, including the Angiotensin-(1-7) peptide and peptidic analogues, agonists and antagonists thereof as modulators of the apoptotic activity for use in gene therapy of diseases as well as organic alterations produced by aging.

13. Use of Mas G-protein-coupled receptor antagonists and agonists according to claim 8, characterized by the use of Mas G-protein-coupled receptor antagonists and agonists, including the Angiotensin-(1-7) peptide and its analogues, agonists and antagonists, either peptidic or non-peptidic, as modulators of the apoptotic activity in the study, prevention and treatment of cardiovascular diseases and their complications, autoimmune diseases, genetic polymorphism consequent diseases, as the DD type of the angiotensin-converting enzyme, associated to reductions in the expression of the Mas receptor, as well as reductions of the plasmatic and tissular levels of the Mas receptor agonists, complications associated to ischemic events in organs and tissues like myocardial infarct, wounds, burns, erythemas, tumors, type-I and type-II diabetes mellitus and its complications, disorders of the male (spermatogenesis, spermatic motility, erectile dysfunction) and female reproductive system and of embryogenesis, respiratory diseases, nephropathies, gastrointestinal disorders, gynecologic disorders, angiogenesis, alopecia, blood diseases and angioplasty (endoluminal prosthesis and post-angioplasty restenosis), disorders of the blood crasis, as for example, post-radiotherapy, disorders of memory and learning and central and peripheral degenerative neuropathies, as well as for use in the study, prevention or treatment of organic alterations produced by aging in warm-blooded animals.

14. Use of Mas G-protein-coupled receptor agonists and antagonists according to claim 2, characterized by the use of Mas G-protein coupled agonists and antagonists, including the Angiotensin-(1-7) peptide and its analogues, agonists and antagonists, either peptidic or non-peptidic, in the study, prevention and treatment of diseases that involve reduction of oxygen reactive species with the consequent endothelial dysfunction, as for example, but not limitative, cardiovascular diseases (high blood pressure, atherosclerosis, thrombosis, myocardial infarct, heart failure and others), renal diseases, plurimetabolic syndrome, erectile dysfunction and diseases of the central nervous system and others.

15. Use of Mas G-protein-coupled receptor agonists and antagonists in the prevention and treatment of diseases that involve reduction of oxygen reactive species with the consequent endothelial dysfunction, as for example, but not limitative, cardiovascular diseases (high blood pressure, atherosclerosis, thrombosis, myocardial infarct, heart failure), renal diseases, plurimetabolic syndrome, erectile dysfunction and diseases of the central nervous system and others, characterized by the use of Mas G-protein-coupled receptor agonists and antagonists, including the Angiotensin-(1-7) peptide and its analogues, agonists and antagonists, either peptidic or non-peptidic, and formulations thereof.

16. Use of Mas G-protein-coupled receptor agonists and antagonists according to claim 1, including the Angiotensin-(1-7) peptide and its analogues, agonists and antagonists, characterized by the use of cDNAs that encode peptide sequences corresponding to Angiotensin-(1-7) and its analogues, agonists and antagonists, for use in gene therapy of diseases that involve reduction of oxygen reactive species with the consequent improvement of the endothelial dysfunction, among them cardiovascular diseases (systemic and pulmonary high blood pressure, hypertensive pregnancy sickness, atherosclerosis, thrombosis, myocardial infarct, heart failure and others), renal diseases, plurimetabolic syndrome, erectile dysfunction, diseases of the central nervous system, vasculitis.

17. Use of Mas G-protein-coupled receptor agonists and antagonists for prevention and treatment of cardiovascular diseases and their complications, autoimmune diseases, genetic polymorphisms consequent diseases, like the DD type of the angiotensin-converting enzyme, associated to reductions in the expression of the Mas receptor as well as reductions of the plasmatic and tissular levels of the Mas receptor agonists, complications associated to ischemic events in organs and tissues like myocardial infarct, wounds, bums, erythemas, tumors, type-I and type-II diabetes mellitus and its complications, disorders of the male (spermatogenesis, spermatic motility, erectile dysfunction) and female reproductive system and of embryogenesis, respiratory diseases, nephropathies, gastrointestinal disorders, gynecologic disorders, angiogenesis, alopecia, blood diseases and angioplasty (endoluminal prosthesis and post-angioplasty restenosis), disorders of the blood era-sis, as for example, post-radiotherapy, memory and learning disorders and central and peripheral degenerative neuropathies, as well as for use in the study, prevention or treatment of organic alterations produced by aging in warm-blooded animals, characterized by the use of Mas G-protein receptor agonists and antagonists, including the Angiotensin-(1-7) peptide and its analogues, agonists and antagonists, either peptidic or non-peptidic, formulated with pharmaceutically or pharmacologically acceptable excipients or carriers, as modulators of the apoptotic activity.

18. Use of Mas G-protein-coupled receptor agonists and antagonists, characterized by the use of Mas G-protein receptor agonists and antagonists, including the Angiotensin-(1-7) peptide and its analogues, agonists and antagonists, either peptidic or non-peptidic and formulations thereof for use either by the oral, intramuscular, intravenous, subcutaneous, topical, transdermic, anal, inhalation (pulmonary, intranasal, intrabuccal) administration routes or as devices that may be implanted or injected for study, prevention and treatment of cardiovascular diseases and their complications, auto-immune diseases, genetic polymorphisms consequent diseases, like the DD type for the angiotensin-converting enzyme, associated to reductions in the expression of the Mas receptor as well as reductions of the plasmatic and tissular levels of the Mas receptor agonists, complications associated to ischemic events in organs and tissues, wounds, burns, erythemas, tumors, type I and type II diabetes mellitus and their complications, disorders of the male (spermatogenesis, spermatic motility, erectile dysfunction) and female reproductive system and of the embryogenesis, respiratory diseases, nephropathies, gastrointestinal disorders, gynecological disorders, angiogenesis, alopecia, blood diseases and angioplasty (endoluminal prosthesis and post-angioplasty restenosis), disorders of the blood crasis, as for example, post-radiotherapy, memory and learning disorders and central and peripheral degenerative neuropathies, as well as for use in the study, prevention or treatment of organic alterations produced by aging in warm-blooded animals.

19. Use of Mas G-protein-coupled receptor agonists and antagonists for prevention and treatment of cardiovascular diseases and their complications, autoimmune diseases, diseases consequent to genetic polymorphism, consequent diseases as the DD type, associated to reductions in the expression of the Mas receptor, as well as reductions of the plasmatic and tissular levels of the Mas receptor agonists, complications associated to ischemic events in organs and wounded tissues, burns, erythemas, tumors, type-I and type-II diabetes mellitus and its complications, disorders of the male (spermatogenesis, spermatic motility, erectile dysfunction) and female reproductive system and of embryogenesis, respiratory diseases, nephropathies, gastrointestinal disorders, gynecologic disorders, angiogenesis, alopecia, blood diseases and angioplasty (endoluminal prosthesis and post-angioplasty restenosis), disorders of the blood crasis, as for example, post-radiotherapy, memory and learning disorders and central and peripheral degenerative neuropathies, as well as for use in the study, prevention or treatment of organic alterations produced by aging in warm-blooded animals, characterized by the use of Mas G-protein-coupled receptor agonists and antagonists, including the Angiotensin-(1-7) peptide and its analogues, agonists and antagonists, either peptidic or non-peptidic, and formulations thereof for use either by the oral, intramuscular, intravenous, subcutaneous, topical, transdermic, anal, inhalation (pulmonary, intranasal, intrabuccal) application routes or as devices that may be implanted or injected.

20. Use of Mas G-protein-coupled receptor agonists and antagonists, including the Angiotensin-(1-7) peptide and its analogues, agonists and antagonists, either peptidic or non-peptidic, and formulations thereof for use either by the oral, intramuscular, intravenous, subcutaneous, topical, transdermic, anal, inhalation (pulmonary, intranasal, intrabuccal) administration routes or as devices that may be implanted or injected, for study, prevention and treatment of skin injuries, wounds, burns, erythemas, tumors, as an auxiliary measure for transplantation of organs, treatment with embryonic, non-embryonic stem cells, re-implantation of organs and other tissues, and other treatments that need temporary or chronic reduction of the apoptotic activity, disorders of the reproductive system (spermatogenesis, spermatic motility, erectile dysfunction), bronchial diseases, nephropathies, gastrointestinal and gynecological disorders, angiogenesis, alopecia, blood diseases and angioplasty (endoluminal prosthesis and post-angioplasty restenosis), disorders of the post-radiotherapy blood crasis, disorders of the memory and of the learning, as well as in the prevention or treatment of organic alterations produced by aging, in warm-blooded animals, characterized by the use of Mas G-protein-coupled receptor agonists and antagonists, including the Angiotensin-(1-17) peptide and its analogous, agonists and antagonists, either peptidic or non-peptidic, formulated with pharmaceutically or pharmacologically acceptable excipients or carriers, as modulators of the apoptotic activity.

21. Use of Mas G-protein-coupled receptor agonists and antagonists according to claim 1, characterized by the use in the treatment of skin injuries, wounds, erythemas, tumors and other treatments that need temporary or chronic reduction of the apoptotic activity, disorders of the reproductive system (spermatogenesis, spermatic motility, erectile dysfunction), bronchial diseases, nephropathies, gastrointestinal and gynecological disorders, angiogenesis, alopecia, blood diseases and angioplasty (endoluminal prosthesis and post-angioplasty restenosis), disorders of the post-radiotherapy blood crasis, memory and learning disorders, as well as in the prevention or treatment of organic alterations produced by aging, in warm-blooded animals.

22. Use of Mas G-protein-coupled receptor agonists and antagonists according to claim 1, characterized by the use as an auxiliary measure for transplantation of organs, treatment with embryonic or non-embryonic stem cells, re-implantation of organs and tissues and other treatments that need temporary or chronic reduction of the apoptotic activity, as well as use in the prevention or treatment of organic alterations produced by aging in warm-blooded animals.

23. Use of Mas G-protein-coupled receptor agonists and antagonists, as an auxiliary measure for organs transplantation, treatment with embryonic or non-embryonic stem cells, reimplantation of organs and tissues and other treatments that need temporary or chronic reduction of the apoptotic activity, as well as use in the prevention or treatment of organic alterations produced by aging in warm-blooded animals, characterized by the use of Mas G-protein-coupled receptor agonists and antagonists, including the Angiotensin-(1-7) peptide and its analogues, agonists and antagonists, either peptidic or non-peptidic, and formulations thereof for use either by the oral, intramuscular, intravenous, subcutaneous, topical, transdermic, anal, inhalation (pulmonary, intranasal, intrabuccal) administration routes or as devices that may be implanted or injected.

24. Use of Mas G-protein-coupled receptor agonists and antagonists for the study, prevention or treatment of skin injuries, wounds, burns, erythemas, tumors, as an auxiliary measure for transplantation of organs, treatment with embryonic, non-embryonic stem cells, reimplantation of organs and tissues, and other treatments that need temporary or chronic reduction of the apoptotic activity, disorders of the reproductive system (spermatogenesis, spermatic motility, erectile dysfunction), bronchial diseases, nephropathies, gastrointestinal and gynecological disorders, angiogenesis, alopecia, blood diseases and angioplasty (endoluminal prosthesis and post-angioplasty restenosis), disorders the post-radiotherapy blood crasis, disorders of the memory and of the learning, as well as in the prevention or treatment of organic alterations produced by aging, in warm-blooded animals, characterized by being use of Mas-G-protein-coupled receptor agonists and antagonists, including the Ang-(1-7) peptide and analogs, agonists and antagonists thereof, either peptidic or non-peptidic, formulated with pharmaceutically or pharmacologically acceptable excipients or carriers, as modulators of the apoptotic activity.

25. Use of Mas G-protein-coupled receptor agonists and antagonists, characterized by the use of Mas G-protein-coupled receptor agonists and antagonists, including the Angiotensin-(1-7) peptide and its analogues, agonists and antagonists, either peptidic or non-peptidic, formulations thereof for use either by the oral, intramuscular, intravenous, subcutaneous, topical, transdermic, anal, inhalation (pulmonary, intranasal, intrabuccal) administration routes or as devices that may be implanted or injected, for study, prevention and treatment in skin injuries, wounds, burns, erythemas, tumors, as an auxiliary measure for transplantation of organs, treatment with embryonic, non-embryonic stem cells, re-implantation of organs and tissues, and other treatments that need temporary or chronic reduction of the apoptotic activity, disorders of the reproductive system (spermatogenesis, spermatic motility, erectile dysfunction), bronchial diseases, nephropathies, gastrointestinal and gynecological disorders, angiogenesis, alopecia, blood diseases and angioplasty (endoluminal prosthesis and post-angioplasty restenosis), disorders of the post-radiotherapy blood crasis, memory and learning disorders, as well as in the prevention or treatment of organic alterations produced by aging, in warm-blooded animals.

26. Use of Mas G-protein-coupled receptor agonists and antagonists, characterized by the use of Mas G-protein-coupled receptor agonists and antagonists, including the Angiotensin-(1-7) peptide and its analogues, agonists and antagonists, either peptidic or non-peptidic, and formulations thereof for use either by the oral, intramuscular, intravenous, subcutaneous, topical, transdermic, anal, inhalation (pulmonary, intranasal, intrabuccal) administration routes or as devices that may be implanted or injected, for study, prevention or treatment of diseases that involve alterations in the muscular differentiation, maturation and regeneration in muscular atrophies like cachexia, cancer, AIDS prolonged restriction to bed due to numberless factors, diabetes, chronic use of corticoids and varied neurological syndromes, traumatisms and degenerative diseases that lead to muscular atrophy, as well as for prevention or treatment of organic alterations produced by aging and as an ergogenic aid.

27. Use of Mas G-protein-coupled receptor agonists and antagonists for prevention and treatment of diseases that involve alterations in the muscular differentiation, maturation and regeneration in muscular atrophies such as: cachexia, cancer, AIDS, prolonged restriction to bed due to number-less factors, diabetes, chronic use of corticoids and varied neurological syndromes, traumatisms and degenerative diseases that lead to muscular atrophy, as well as for prevention or treatment of organic alterations produced by aging and as an ergogenic aid, characterized by the use of Mas G-protein-coupled agonists and antagonists, including the Angiotensin-(1-7) peptide and its analogues, agonists and antagonists, either peptidic or non-peptidic, and formulations thereof for use either by the oral, intramuscular, intravenous, subcutaneous, topical, transdermic, anal, inhalation (pulmonary, intranasal, intrabuccal) administration routes or as devices that may be implanted or injected.

28. Use of Mas G-protein-coupled agonists and antagonists for prevention and treatment of diseases that involve reduction of oxygen reactive species with the consequent improvement in the endothelial dysfunction, among them cardiovascular diseases (systemic and pulmonary high blood pressure, hypertensive pregnancy sickness, atherosclerosis, thrombosis, myocardial infarct, heart failure and others), renal diseases, plurimetabolic syndrome, erectile dysfunction, diseases of the central nervous system, vasculitis, characterized by the use of Mas G-protein-coupled receptor agonists and antagonists, including the Angiotensin-(1-7) peptide and its analogues, agonists and antagonists, either peptidic or non-peptidic, either by the oral, intramuscular, intravenous, subcutaneous, topical, transdermic, anal, inhalation (pulmonary, intranasal, intrabuccal) administration routes or as devices that may be implanted or injected.

29. Use of Mas G-protein-coupled receptor agonists and antagonists, characterized by the use of Mas G-protein-coupled receptor agonists and antagonists, including the Angiotensin-(1-7) peptide and its analogues, agonists and antagonists, either peptidic or non-peptidic, and formulations thereof for use either by the oral, intramuscular, intravenous, subcutaneous, topical, transdermic, anal, inhalation (pulmonary, intranasal, intrabuccal) administration routes or as devices that may be implanted or injected, for study, prevention and treatment of diseases that involve reduction of oxygen reactive species with the consequent improvement of the endothelial dysfunction, among them cardiovascular diseases (systemic and pulmonary high blood pressure, hypertensive pregnancy sickness, atherosclerosis, thrombosis, myocardial infarct, heart failure and others), renal diseases, plurimetabolic syndrome, erectile dysfunction, diseases of the central nervous system, vasculitis.

30. Use of Mas G-protein-coupled receptor agonists and antagonists, for study, prevention or treatment of diseases that involve alterations in the muscular differentiation, maturation and regeneration in muscular atrophies such as: cachexia, cancer, AIDS, prolonged restriction to bed due to numberless factors, diabetes, chronic use of corticoids and varied neurological syndromes, traumatisms and degenerative diseases that lead to muscular atrophy, as well as for prevention or treatment of organic alterations produced by aging and as an ergogenic aid, characterized by the use of Mas G-protein-coupled receptor agonists and antagonists, either peptidic or non-peptidic, including the Angiotensin-(1-7) peptide and its analogues, agonists and antagonists, either peptidic or non-peptidic, formulated with pharmaceutically or pharmacologically acceptable excipients or carriers.

31. Use of Mas G-protein-coupled receptor agonists and antagonists, for study, prevention or treatment of diseases that involve reduction of oxygen reactive species with the consequent improvement of the endothelial dysfunction, among them cardiovascular diseases (systemic and pulmonary high blood pressure, hypertensive pregnancy sickness, atherosclerosis, thrombosis, myocardial infarct, heart failure, and others), renal diseases, plurimetabolic syndrome, erectile dysfunction, diseases to the central nervous system, vasculitis, characterized by the use of Mas G-protein-coupled receptor agonists and antagonists, including the Angiotensin-(1-7) peptide and its analogues, agonists and antagonists, either peptidic or non-peptidic, formulated with pharmaceutically or pharmacologically acceptable excipients or carriers.
